(19) United States
(12) Patent Application Publication
(10) Pub. No.: US 2024/0216667 A1
(43) Pub. Date: Jul. 4, 2024

(54) PERITONEAL DIALYSIS CAPS, SYSTEMS & METHODS

(71) Applicant: ICU Medical, Inc., San Clemente, CA (US)

(72) Inventors: Robert J. Ziebol, Shoreview, MN (US); Matthew David Beilke, Plymouth, MN (US)

(21) Appl. No.: 18/608,049

(22) Filed: Mar. 18, 2024

Related U.S. Application Data
(63) Continuation of application No. 18/320,769, filed on May 19, 2023, now Pat. No. 11,944,776.
Application No. 18/320,769 is a continuation of application No. PCT/US2021/062079, filed on Dec. 6, 2021.
(60) Provisional application No. 63/122,470, filed on Dec. 7, 2020.

(51) Int. Cl. A61M 39/20; A61M 39/16

(52) U.S. Cl. CPC A61M 39/20; A61M 39/162; A61M 39/165

(57) ABSTRACT

A cap for a medical connector, the cap including a body having a closed proximal end and an open distal end, an interior volume within the body, an elongate member comprising an antimicrobial extending from the proximal end of body axially through at least a portion of the interior volume, the elongate member, threads for securing the cap to a medical connector, and a radially inwardly facing sealing surface on the cap, the inwardly facing sealing surface located distal to the threads and providing a liquid-tight seal between the cap and the medical connector when the cap is installed on the medical connector.

### PRIORITY CLAIM AND INCORPORATION BY REFERENCE

The present application is a continuation of U.S. application Ser. No. 18/320,769 filed May 19, 2023, which is a continuation of PCT Application No. 2021/062,079, filed on Dec. 6, 2021, which claims the benefit under 35 U.S.C. § 119(e) to U.S. Provisional Application No. 63/122,470, filed on Dec. 7, 2020, which are incorporated by reference herein in their entirety. Any and all applications for which a foreign or domestic priority claim is identified in the Application Data Sheet as filed with the present application are hereby incorporated by reference under 37 CFR 1.57 for all purposes and for all that they contain.

### FIELD

Embodiments disclosed herein relate to peritoneal dialysis devices and methods, including peritoneal dialysis caps, cap systems for use during peritoneal dialysis, and methods for administering antimicrobial substances to peritoneal dialysis devices.

### BACKGROUND

Infusion devices, such as catheters and on-catheter devices, are commonly used in providing modern medical care to patients. For example, catheters such as hemodialysis catheters, peritoneal dialysis catheters, peripherally inserted central catheters, midline catheters, and drainage catheters are all commonly used in providing modern medical care to patients. Other infusion devices used in providing medical care include needleless connectors, intravenous (IV) administration sets, peritoneal dialysis lines, transfer sets, syringes, valves and filters.

These infusion devices are useful for treating various medical conditions. For example, peritoneal catheters allow patients with renal disease to have waste and fluid removed from their bodies. Thus, catheters and other infusion devices make critical medical care possible and are often essential to providing improved health care outcomes.

However, long-term use of catheters has a serious drawback in that a significant percentage of catheters fail due to infection, resulting in elevated mortality rates and significantly increased healthcare costs associated with treatment. Furthermore, infections are a leading cause of death in the United States, and many of those infections are attributable to infusion devices. The mortality rate associated with such infections is considerable. Therefore, a need exists for a solution to reduce infections relating from the use of infusion devices.

### SUMMARY OF SOME EXEMPLIFYING EMBODIMENTS

The systems, methods, and devices of this disclosure each have several innovative aspects, implementations, or aspects, no single one of which is solely responsible for the desirable attributes disclosed herein. Nothing in this specification is essential or indispensable. Any structure, material, component, method, or step described and/or illustrated in any embodiment in this specification can be provided by itself and/or combined with any other structure, material, component, method, or step described and/or illustrated in any other embodiment in this specification or known in the art. All relative sizes and dimensions of parts and components illustrated in the drawings are intended to form part of this disclosure but should not be used to limit the scope of any claim unless recited in such claim. Any words in this specification that are generally associated with circles or circular structures, such as the words radius, radial, diameter, or circumference, should be understood to also apply to non-circular structures in analogous ways, such as denoting cross-sectional widths or perimeters.

Infection-causing organisms are ever present in the environment; they live on patients' skin and can survive and be transmitted in air and water. Conventional medical device connectors and caps, such as male and female connectors with tapered luers, have a threaded region along with a tapered scaling region, such as an overlapping scaling region of the tapered portions of male and female connectors. The overlapping sealing regions seal fluid inside the medical device and keep air and organisms out. However, our testing shows that organisms can still migrate through the threaded region and penetrate a portion of the way into the sealing region. This results in organisms being present along the walls of portions of the devices. When the male and female connectors are separated from one another, some organisms can remain on the walls of the male and female connectors. The next time a connection is made, some of the organisms can be pushed past the scaling surface and into the fluid path. Once organisms are in the fluid path they can multiply, spread, and cause an infection. Peritoneal dialysis transfer sets can be used to make fluid connections between a peritoneal dialysis catheter and a patient line, for instilling and removing dialysate from the peritoneal cavity.

Disclosed herein are embodiments of a cap for a medical connector that can include a body having a closed proximal end and an open distal end, an interior volume within the body, an elongate member comprising an antimicrobial extending from the proximal end of body axially through at least a portion of the interior volume, the elongate member, threads for securing the cap to a medical connector, and a radially inwardly facing sealing surface on the cap, the inwardly facing sealing surface located distal to the threads and providing a seal, such as a liquid-tight and/or an air-tight seal, between the cap and the medical connector when the cap is installed on the medical connector.

Any embodiments of the caps, methods, and systems disclosed herein can include, in additional embodiments, one or more of the following steps, features, components, and/or details, in any combination with any of the other steps, features, components, and/or details of any other embodiments disclosed herein: wherein the medical connector is part of a peritoneal dialysis transfer set; wherein the radially inwardly facing sealing surface on the cap provides a seal (e.g., air-tight and/or liquid-tight) between the cap and the medical connector when the cap is installed on the medical connector by constricting around an outer surface of the medical connector; further comprising a distally facing sealing surface on the cap, wherein the distally facing sealing surface is located distal to the threads, and the distally facing sealing surface provides a second liquid-tight seal between the cap and the medical connector when the cap is installed on the medical connector; wherein the distally facing sealing surface is located at a distal end of the cap and has a generally planar surface that is approximately perpendicular to a longitudinally axial centerline of the cap; wherein all sealing surfaces are located in a distal portion of the body of the cap; wherein the cap is configured to overlap a portion of an outer surface of the medical connector and to create a seal against the portion of the outer surface of the medical connector that is overlapped; comprising a porous element in the interior volume within the body, the porous element being dry and being free of any antimicrobial substances prior to installation on a medical connector; wherein the porous element is configured to retain a fluid exiting the medical connector upon installation of the cap on the transfer set; wherein the porous element encloses a volume of at least about 75%, or less than or equal to about 75%, or between about 75% to about 125%, or approximately 125%, or less than or equal to about 125%, or at least about 125%, of a volume of liquid that can be displaced by the elongate member as the elongate member is advanced into the medical connector; wherein the porous element is sized and configured to contact an end of the medical connector when the cap is engaged with the medical connector; wherein the elongate member does not form a fluid seal within a lumen of the medical connector; and/or wherein the antimicrobial comprises chlorhexidine acetate. Also disclosed herein are embodiments of a kit that can include a cap for a medical connector of any embodiments disclosed herein and a base element, wherein the cap is coupled with the base element.

Disclosed herein are embodiments of a cap for a medical connector that can include a body having a closed proximal end and an open distal end, an interior volume within the body, an elongate member extending from the proximal end of body axially through at least a portion of the interior volume, the elongate member comprising a dry antimicrobial on a surface thereof, threads for securing the cap to a medical connector, a porous element in the interior volume within the body, wherein the porous element is not saturated with or substantially free of an antimicrobial before use, and a radially inwardly facing sealing surface on the cap. In some embodiments, the inwardly facing scaling surface can be located distal to the threads and can provide a liquid-tight seal between the cap and the medical connector when the cap is installed on the medical connector. In some embodiments, the porous element can be at least about 95% free of an antimicrobial before use. This can mean that the porous element can have about 5% or less of a total amount of an antimicrobial that the porous element can be capable of retaining within the porous element. In some embodiments, the porous element can be at least about 90% free of an antimicrobial before use. This can mean means that the porous element can have 10% or less of a total amount of an antimicrobial that the porous element can support.

Also disclosed herein are embodiments of a method of delivering an antimicrobial to a medical connector that can include advancing a cap into engagement with a medical connector having a lumen having an inner wall, an outer wall having an exterior surface, and a proximal end face, advancing an elongate member of the cap into the lumen so as to wet a dry antimicrobial coated on an outside surface of the elongate member to dissolve the antimicrobial into a liquid within the medical connector, and sealing the cap against an outwardly facing surface of the medical connector with a sealing surface located at a distal end of the cap.

Disclosed herein are embodiments of a cap or set of caps (collectively referred to herein as a cap) for a medical connector for peritoneal dialysis that can include a partially enclosed interior space (also referred to herein as an interior space), an elongate member positioned at least partially within the partially enclosed interior space, an antimicrobial on or in the elongate member, threads on the cap for securing the cap to the medical connector, and a sealing surface on the cap. In some embodiments, the scaling surface can be located distal to the threads and can provide a liquid-tight seal between the cap and the medical connector when the cap is installed on the medical connector.

Disclosed herein are embodiments of a cap for a medical connector for peritoneal dialysis that can include a partially enclosed interior space, an elongate member positioned at least partially within the partially enclosed interior space, an antimicrobial on or in the elongate member, threads on the cap for securing the cap to the medical connector, and a sealing surface on the cap. In some embodiments, the sealing surface can be located distal to the threads and can provide a first sealing capacity when installed on the medical connector. The first sealing capacity can be more than 50 percent of a total scaling capacity between the cap and the medical connector when the cap is installed on the medical connector.

Disclosed herein are embodiments of a cap for a medical connector for peritoneal dialysis that can include a partially enclosed interior space, an elongate member positioned at least partially within the partially enclosed interior space, an antimicrobial on or in the elongate member, threads on the cap for securing the cap to the medical connector, and a sealing surface on the cap. In some embodiments, the sealing surface can have an inner diameter that is greater than an inner diameter of the threads on the cap.

Disclosed herein are embodiments of a cap for a medical connector for peritoneal dialysis that can include an elongate member, an antimicrobial on or in the elongate member, threads on the cap for securing the cap to the medical connector, the threads on the cap having an inner diameter, and a sealing surface on the cap, the sealing surface having an inner diameter greater than the inner diameter of the threads on the cap.

Disclosed herein are embodiments of a cap for a medical connector for peritoneal dialysis that can include a partially enclosed interior space, an elongate member positioned at least partially within the partially enclosed interior space, an antimicrobial on or in the elongate member, threads on the cap for securing the cap to the medical connector, and a sealing surface on the cap. In some embodiments, the sealing surface can be located distal to the threads and can be configured to provide a seal between the cap and the medical connector. In some embodiments, the cap can be configured to not form an additional seal with the medical connector when installed on the medical connector.

Disclosed herein are embodiments of a cap for delivering an antimicrobial to a medical connector that can include an elongate member, an antimicrobial on or in the elongate member, threads having an inner diameter, and a sealing surface. The threads can be configured to secure the cap to the medical connector, and the sealing surface can have an inner diameter greater than the inner diameter of the threads.

Disclosed herein are embodiments of a cap for delivering an antimicrobial to a medical connector that can include an elongate member, an antimicrobial on or in the elongate member, threads configured to secure the cap to the medical connector, and a sealing surface, wherein the elongate member is located at least partially proximal to the sealing surface.

Disclosed herein are embodiments of a cap for a medical connector that can include a partially enclosed interior space, an elongate member at least partially within the partially enclosed interior space, an antimicrobial on or in the elongate member, and a compressible porous element (also referred to herein as a porous material) positioned near a proximal portion of the elongate member, the compressible porous element substantially free of an antimicrobial before use.

Disclosed herein are embodiments of a cap for a medical connector that can include a proximal end and a distal end, with an opening on the distal end leading to an interior of the cap, treads located in a partially enclosed interior space of the cap, an elongate member at least partially within the partially enclosed interior space of the cap, a dry antimicrobial on the elongate member, and a sealing surface located entirely distal to threads on the cap.

Disclosed herein are embodiments of a cap for a medical connector that can include an elongate member configured to be inserted into a lumen of the medical connector, an antimicrobial on or in the elongate member, and a sealing surface located distal to at least a portion of the elongate member and configured to form a fluid tight seal.

Disclosed herein are embodiments of a cap for a medical connector that can include an elongate member configured to be inserted into a lumen of the medical connector, an antimicrobial on or in the elongate member, and a sealing surface configured to form a fluid tight seal on an outer surface of the medical connector.

Disclosed herein are embodiments of a cap for a medical connector that can include an elongate member configured to be inserted into a lumen of the medical connector, an antimicrobial on or in the elongate member, and a sealing surface having an internal diameter that is larger than a thread outer diameter of the medical connector.

Disclosed herein are embodiments of a cap for a medical connector that can include a proximal end and a distal end, with an opening on the distal end, an elongate member at least partially within an interior of the cap, a dry antimicrobial on the elongate member, and a sealing surface on the distal end of the cap, wherein the sealing surface is configured to prevent venting after the cap is installed.

Disclosed herein are embodiments of a cap for a medical connector that can include a proximal end and a distal end, with an opening on the distal end, an elongate member at least partially within an interior of the cap, a dry antimicrobial on the elongate member, and a porous element positioned at a proximal end of the elongate member, wherein, after installation of the cap onto the medical connector, the dry antimicrobial can be configured to move within a lumen region between the porous element and a clamp of the medical device.

Disclosed herein are embodiments of a method of in-situ formation of an antimicrobial porous element including providing a cap having a proximal end and a distal end, an opening on the distal end, an elongate member at least partially within an interior of the cap, a dry antimicrobial on the elongate member, and a compressible porous element substantially free of antimicrobial, providing a medical device having a lumen, and inserting the elongate member into a liquid contained within the lumen of the medical device such that the dry antimicrobial dissolves into the liquid to create an antimicrobial liquid and wets the porous element with the antimicrobial liquid.

Disclosed herein are embodiments of a method of in-situ formation of an antimicrobial porous element that can include providing a cap having: a proximal end and a distal end, an opening on the distal end, an elongate member at least partially within an interior of the cap, the elongate member having a volume, a dry antimicrobial on the elongate member, and a compressible porous element partially surrounding a portion of the elongate member, and inserting the elongate member into a liquid such that the volume of the elongate member displaces a portion of the liquid through the compressible porous element and at least a portion of the dry antimicrobial dissolves into the liquid to form an antimicrobial liquid, and at least a portion of the antimicrobial liquid makes contact with the porous element. In some embodiments, the compressible porous element can be substantially free of antimicrobial and can at least partially contain air.

Disclosed herein are embodiments of a method of cleaning a proximal end of a dialysis transfer set that can include providing a cap having a proximal end and a distal end, with an opening on the distal end, an elongate member at least partially within the interior of the cap, a dry antimicrobial on the elongate member, and a compressible porous element partially surrounding a portion of the elongate member, and installing the cap on the transfer set. In some embodiments, the compressible porous element can be compressed during installation by a swiping motion on the proximal end of the transfer set.

Disclosed herein are embodiments of a method of installing a cap to a medical connector that can include providing a cap having a proximal end and a distal end, an opening on the distal end, an elongate member at least partially within the interior of the cap, a dry antimicrobial on the elongate member, a dry compressible porous element partially surrounding a portion of the elongate member, and installing the cap on the transfer set. In some embodiments, the compressible porous element can be substantially free of antimicrobial. In some embodiments, when inserted into the lumen, the elongate member can displace liquid in a lumen of the transfer set such that air transfers through the porous element and liquid is retained in the compressible porous element.

Disclosed herein are embodiments of a capping system for a medical connector that can include a base element containing a finger retainer on a distal end of the base element, a cap having a partially enclosed interior space, a proximal end and distal end, and a heat shrink band at a distal end of the cap and the proximal end of the base element, and providing a barrier to the interior of the cap. Disclosed herein are embodiments of a cap system for a medical connector that can include a base element containing a finger retainer on the distal end of the cap system, and a cap having a partially enclosed interior space, the cap having a proximal end and distal end. Also disclosed herein are embodiments of a cap system for a medical connector that can include a base element containing a finger retainer on a distal end of the cap system, a cap, and a heat shrink band at the distal end of the cap providing a barrier to a partially enclosed interior space of the cap.

Disclosed herein are embodiments of a method of retaining a cap on a base such that the cap cannot be reinstalled on the base. The method can include providing a cap system including a cap and removing the cap from the base such that the heat shrink band is retained on the base but the heat shrink band contracts to prevent reinstallation onto the base. The cap can include a partially enclosed interior space, a proximal end, and a distal end. The cap system can further include a base element containing a finger retainer on the proximal end of the cap and a heat shrink band at a distal end of the cap providing a barrier to the partially enclosed interior space of the cap. The cap can be secured to the base element by the heat shrink band, for example at or adjacent to a distal end of the cap.

Disclosed herein are embodiments of a method of delivering an antimicrobial to a medical connector that can include providing a medical connector having a lumen, an inner wall and a proximal end face, providing a cap having a proximal end and a distal end, with an opening on the distal end, an elongate member at least partially within an interior of the cap, and a dry antimicrobial on the elongate member, a dry compressible porous element substantially free of antimicrobial, and inserting the elongate member into a liquid contained within the lumen such that the dry antimicrobial dissolves into the liquid to create an antimicrobial liquid within the lumen and the porous element. In some embodiments, the antimicrobial liquid within the lumen can contact the inner wall, and the antimicrobial within the porous element can contact the proximal end face.

Disclosed herein are embodiments of a cap for a medical connector substantially as described herein or shown in the accompanying drawings. Also disclosed herein are embodiments of a method of using a cap for a medical connector substantially as described herein or shown in the accompanying drawings.

Any embodiments of the caps, methods, and systems disclosed herein can include, in additional embodiments, one or more of the following steps, features, components, and/or details, in any combination with any of the other steps, features, components, and/or details of any other embodiments disclosed herein: wherein medical connector is part of a peritoneal dialysis transfer set; wherein the sealing surface is located in an exterior direction from the threads; wherein the cap further includes a proximal cavity in the partially enclosed interior space; wherein a proximal cavity includes a portion of interior proximal to proximal end of transfer set when the cap is installed on a transfer set; wherein a porous element is included within the proximal cavity; wherein the porous element is dry prior to installation on a transfer set; wherein the porous element is free of any antiseptic or antimicrobial substance or other substance, prior to installation; wherein the porous element is configured to retain a fluid exiting the medical connector upon installation of the cap on the medical connector; wherein the proximal cavity encloses a volume greater than the volume of the elongate member displacing volume; wherein the porous element in the proximal cavity encloses a volume from 75 to 125 percent of the elongate member displacing volume; wherein the porous element in the proximal cavity encloses a volume from 50 to 150 percent of the volume of the elongate member displacing volume; wherein the proximal cavity includes one or more dividers to form sub-cavities; wherein the proximal cavity provides a fluid flow path for fluid to exit a lumen of the medical connector during installation of the cap; wherein the elongate member is contained entirely within the partially enclosed interior space of the cap; wherein the elongate member extends out a distal end of the partially enclosed interior space; wherein the sealing surface faces radially inward and is located in an exterior direction from the threads; wherein the cap further includes a distal planar sealing surface which faces distally and is located in an exterior direction from the threads; wherein the cap further includes a distal planar scaling surface and a distal radial sealing surface, the sealing surfaces located in an exterior direction from the threads; wherein the cap further includes a distal oblique sealing surface in an exterior direction from the threads; wherein the medical connector has a proximal end and the elongate member has a displacing volume defined as a volume of the elongate member that is distal to the proximal end of the medical connector when the cap is fully installed on the medical connector; wherein the medical connector has a proximal end and the proximal cavity can include a volume of the partially enclosed interior space of the cap proximal to the proximal end of the medical connector when the cap is installed on a medical connector; and/or wherein the medical connector has a proximal end, and the partially enclosed interior space has a distal cavity that can include volume of the partially enclosed interior space distal to the proximal end of the medical connector and proximal to a seal between the cap and medical connector and radially outward from an outer wall of a transfer set when the cap is installed on the medical connector.

Any embodiments of the caps, methods, and systems disclosed herein can include, in additional embodiments, one or more of the following steps, features, components, and/or details, in any combination with any of the other steps, features, components, and/or details of any other embodiments disclosed herein: wherein the elongate member does not form a fluid seal within a lumen of the medical connector; wherein the cap is configured to not a seal within a lumen of the medical connector; wherein the cap further includes a cap retention flange for securing the cap to a base; wherein a cap for a medical connector has a partially enclosed interior space, an elongate member positioned at least partially within the partially enclosed interior space, an antimicrobial on or in the elongate member, threads on the cap for securing the cap to the medical connector, and a sealing surface on the cap, the sealing surface located distal to the threads and providing a primary seal between the cap and the medical connector when the cap is installed on the medical connector; wherein the sealing surface is located in an exterior direction from the threads; wherein the cap further includes a proximal cavity in the partially enclosed interior space; wherein the cap can include a porous element within the proximal cavity; wherein the elongate member is contained entirely within the partially enclosed interior space of the cap; wherein the first sealing capacity is more than 75 percent of a total sealing capacity between the cap and the medical connector when the cap is installed on the medical connector; wherein the first sealing capacity is more than 90 percent of a total sealing capacity between the cap and the medical connector when the cap is installed on the medical connector; wherein the first sealing capacity is more than 95 percent of a total sealing capacity between the cap and the medical connector when the cap is installed on the medical connector; and/or wherein the cap is configured to not a seal within a lumen of the medical connector

Any embodiments of the caps, methods, and systems disclosed herein can include, in additional embodiments, one or more of the following steps, features, components, and/or details, in any combination with any of the other steps, features, components, and/or details of any other embodiments disclosed herein: wherein the sealing surface is a primary sealing surface; wherein the cap is configured to not a seal within a lumen of the medical connector; wherein the sealing surface is located distal to the threads and providing a first sealing capacity when installed on the medical connector, the first sealing capacity is more than 50 percent of a total sealing capacity between the cap and the medical connector when the cap is installed on the medical connector; wherein the sealing surface is located distal to the threads and providing a first sealing capacity when installed on the medical connector, the first sealing capacity is more than 75 percent of a total scaling capacity between the cap and the medical connector when the cap is installed on the medical connector; wherein the sealing surface is located in an exterior direction from the threads; wherein the cap further includes a proximal cavity; wherein further can include a porous element within the proximal cavity; wherein the elongate member is contained entirely within a partially enclosed interior space of the cap; wherein the cap further includes a proximal cavity in the partially enclosed interior space; wherein further can include a porous element within the proximal cavity; wherein the porous element encloses a volume from 75 to 125 percent of the elongate member displacing volume; wherein the porous element encloses a volume from 50 to 150 percent of the volume of the elongate member displacing volume; wherein further can include a silver-based antimicrobial; wherein the silver-based antimicrobial is coated onto portions of the cap; wherein the silver-based antimicrobial is integrally formed into at least a portion of the cap; wherein the silver-based antimicrobial includes a silver salt; wherein the silver-based antimicrobial includes a silver complex; wherein the silver-based antimicrobial includes silver ions; wherein the silver-based antimicrobial includes silver nano particles; wherein the silver-based antimicrobial includes a silver nano layer; wherein the silver-based antimicrobial includes pentasilver hexaoxoiodate, silver-sulfadiazine, silver trifluoroacetate, silver nitrate, silver stearate, and combinations thereof; wherein the antimicrobial includes chlorhexidine acetate; wherein the cap includes chlorhexidine acetate and a silver-based antimicrobial.

### BRIEF DESCRIPTION OF THE FIGURES

FIG. 1 is a schematic illustration of a patient undergoing peritoneal dialysis, showing a peritoneal catheter extending into a peritoneal cavity into which a dialysis solution is injected and then removed.

FIG. 2 is a perspective view of a peritoneal dialysis transfer set.

FIG. 3 is a cross-sectional view along a longitudinal centerline of the peritoneal dialysis transfer set of FIG. 2.

FIG. 4 is a perspective view of an embodiment of a cap combination set including a peritoneal dialysis cap and a base element.

FIG. 5 is a front view of the embodiment of the cap combination set shown in FIG. 4.

FIG. 6 is a cross-sectional view of the embodiment of the cap combination set shown in FIG. 4, taken along line L-L as shown in FIG. 5.

FIG. 7 is a side view of the embodiment of the cap combination set shown in FIG. 4.

FIG. 8 is a cross-sectional view of the embodiment of the cap combination set shown in FIG. 4, taken along line K-K as shown in FIG. 7.

FIG. 9 is a side view of an embodiment a peritoneal dialysis cap.

FIG. 10 is a cross-sectional view of the embodiment of the peritoneal dialysis cap set shown in FIG. 9, taken along line J-J as shown in FIG. 9.

FIG. 11 is a side view of an embodiment of a peritoneal dialysis cap base.

FIG. 12 is a cross-sectional view of the embodiment of the peritoneal dialysis cap base element shown in FIG. 11, taken along line D-D as shown in FIG. 11.

FIG. 13 is a side view of an embodiment of a peritoneal dialysis cap being installed on a hub or end portion of a transfer set.

FIG. 14 is a cross-sectional view of the peritoneal dialysis cap being installed on a hub or end portion of a transfer set as shown in FIG. 13, taken along line H-H as shown in FIG. 13.

FIG. 15 is a side view of an embodiment of a peritoneal dialysis cap installed on a hub or end portion of a transfer set.

FIG. 16 is a cross-sectional view of the embodiment of the peritoneal dialysis cap installed on a hub or end portion of a transfer set as shown in FIG. 15, taken along line I-I as shown in FIG. 15.

FIG. 17 is a cross-sectional view of an embodiment of a peritoneal dialysis cap installed on a hub or end portion of a transfer set in accordance with various embodiments herein, showing proximal/distal orientations and interior/exterior orientations.

FIG. 18 is a perspective view of an embodiment of a peritoneal dialysis cap base element.

FIG. 19 is a cross-sectional view of an embodiment of a peritoneal dialysis cap and base element.

FIG. 20 is a perspective view of another embodiment of a peritoneal dialysis cap base element.

FIG. 21 is a perspective view of another embodiment of a peritoneal dialysis cap base element.

FIG. 22 is a perspective view of another embodiment of a peritoneal dialysis cap base element.

FIG. 23 is a perspective view of another embodiment of a peritoneal dialysis cap base element made in accordance with various embodiments herein.

### DETAILED DESCRIPTION

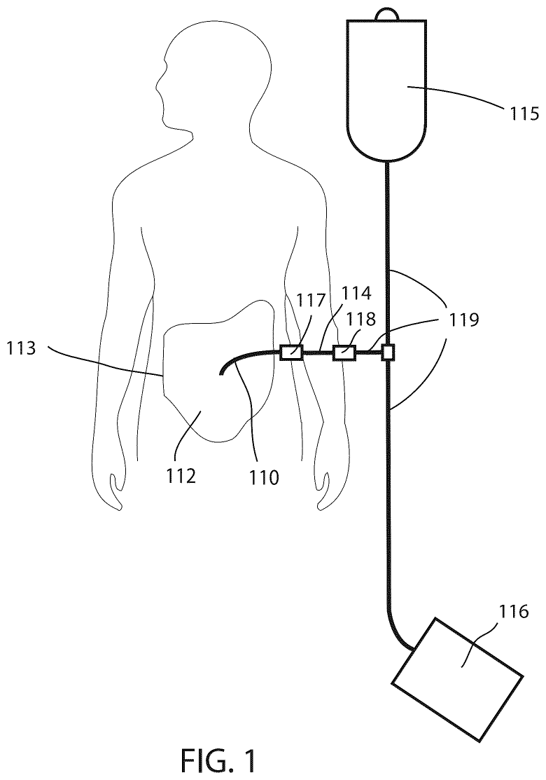

Referring now to the drawings, FIG. 1 is a schematic diagram of a patient undergoing peritoneal dialysis, showing a peritoneal catheter 110 extending into a peritoneal cavity 112 (surrounded by peritoneum 113) of a patient, into which a dialysis solution from source bag 115 flows into the patient. The dialysis solution can later then be drained into drain bag 116. The peritoneal catheter 110 can be in fluid communication with the bags 116 and 115 by means of a transfer set 114 and an infusion set 119. Couplings 117 and 118 can be positioned on either end of the transfer set 114. In some examples, the transfer set 114 can be the peritoneal dialysis transfer set 119 as described.

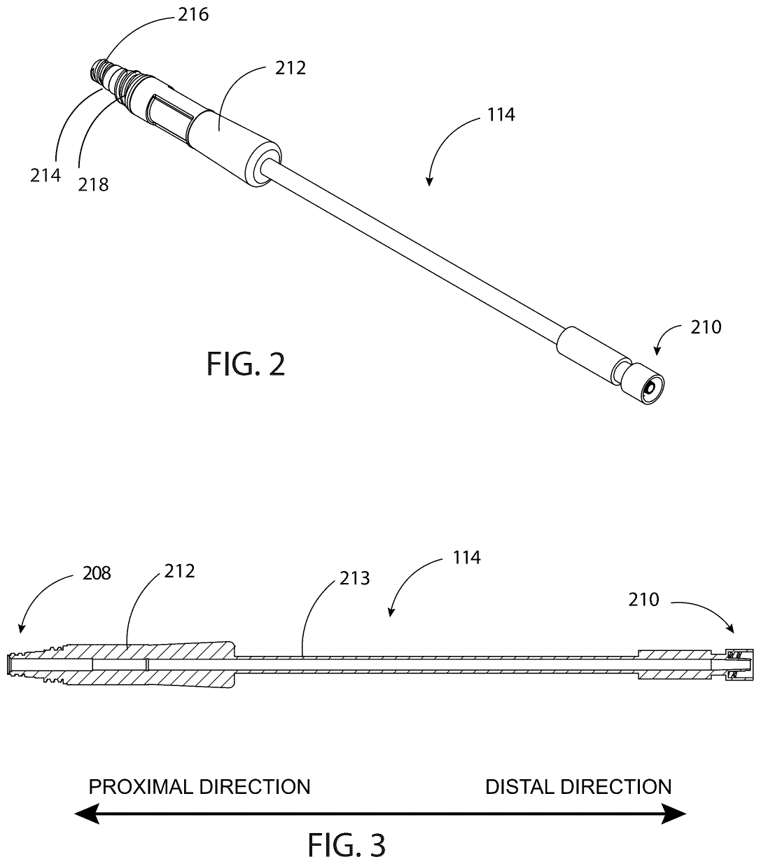

FIG. 2 is a perspective view of a peritoneal dialysis transfer set 114, and FIG. 3 is a section-view of the peritoneal dialysis transfer set 114. The peritoneal dialysis transfer set 114 can include tubing 213 extending from a proximal end 208 of the transfer set 114 to the distal end 210. At the proximal end 208 of the transfer set 114 can be a threaded male connector 214 with first male threads 216 and second male threads 218. The threaded male connector 214 is a proximal end portion of the peritoneal dialysis transfer set 110.

During the exchange process, the waste dialysis solution can flow from the peritoneal cavity 112 to the coupling 117 and transfer set 114, then through the coupling 118 and finally through the lower portion of the infusion set 119 into the drain bag 116. After the exchange process is complete, the infusion set 119 can be separated at coupling 118 from transfer set 114 and the female connector of the transfer set 114 can be capped until the next dialysis solution exchange is initiated (not shown). Thus, in a typical peritoneal dialysis, the exchange process can be initiated by removing a cap from the distal end 210 of the transfer set 114 and then by joining to the infusion set 119 to form the coupling 118. This process can be reversed at the end of the exchange process by removing the infusion set 116 at coupling 118 and installing a new cap.

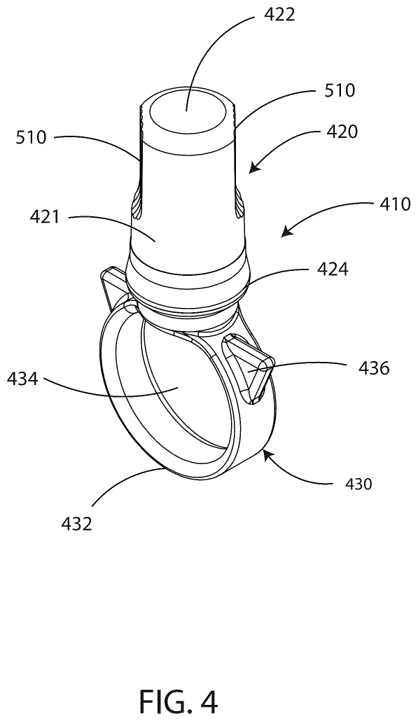

FIG. 4 is a perspective view of an embodiment of a cap combination set 410 comprising a peritoneal dialysis cap 420 and a base element 430. Some embodiments of the peritoneal dialysis cap 420 can have a proximal end 422 and a distal end 424. The base element 430 can include a ring 432 sized to receive a finger of a user, with a hole 434 through the ring 432. In addition, in the embodiment depicted, the base element 430 can include a pair of side extensions 436 that can restrict rotation of the base element 430 when the base element 430 is positioned on a user's hand. For example and without limitation, in some embodiments, a user can press against one or both of the side extensions 436 to increase a torque force applied to the base element 430. In some embodiments, the side extensions 436 can have a pointed or triangular shape, as shown, a rounded shape, or any other desired shape.

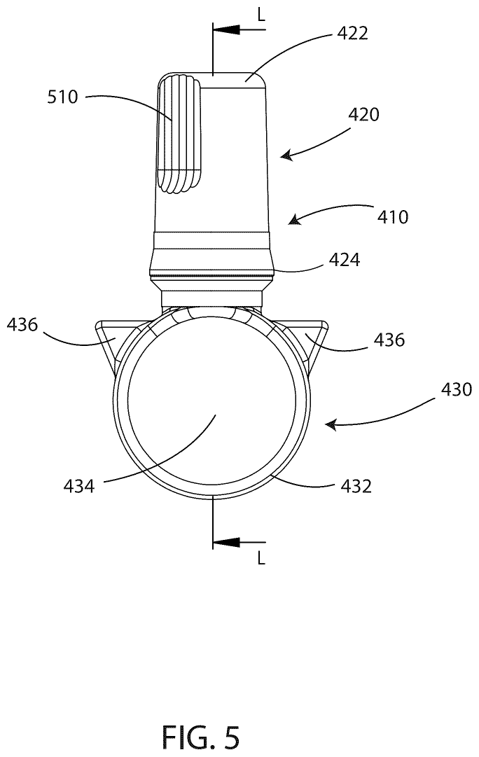

In any embodiments disclosed herein, the cap 420 can have one or more gripping portions 510 on an outside surface of the body 421 of the cap 420. In some embodiments, the gripping portion(s) 510 can be positioned at or adjacent to the proximal end 422 of the cap 420 and can extend along a length of an outside surface of the cap in an axial direction. As shown, some embodiments of the cap 420 can have two gripping portions 510 formed in the body 421 of the cap 420 that are mutually opposed. With reference to FIG. 5, in some embodiments, the gripping portion 510 can have channels, ridges, roughened texture, or other features configured to improve the grip of a user's fingers on the cap 420 during use of the cap 420.

FIG. 5 is a side view of the embodiment of the cap combination set 410 shown in FIG. 4, and can include the peritoneal dialysis cap 420 and the base element 430. The peritoneal dialysis cap 420 can have a proximal end 422 and a distal end 424. The base element 430 can include a ring 432 sized to receive the finger of a user, with a hole 434 through the ring 432. In addition, in the embodiment depicted, the base element 430 can include a pair of side extensions 436, as described above.

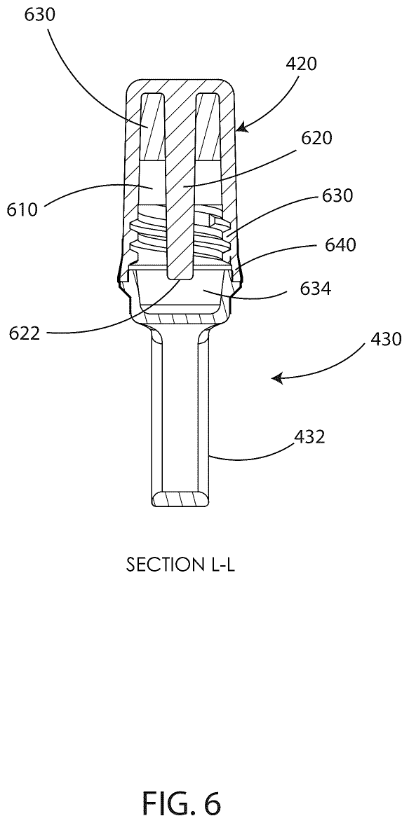

FIG. 6 is a cross-sectional view of the embodiment of the cap combination set 410 containing a peritoneal dialysis cap 420 and base element 430. The base element 430 can include a ring 432 sized to receive the finger of a user, with a hole 434 through the ring 432. Some embodiments of the peritoneal dialysis cap 420 can include an internal volume 610 from which an elongate member 620 can extend, the elongate member having a distal end 622. Some embodiments of the cap 420 can have a porous element 630 which can include a sponge material, an open cell foam material, or other suitable dry foam or dry porous material. In some embodiments, the porous element 630 can have an opening therein configured to receive the elongate member 622 therein. The porous element 630 can be positioned so as to abut against a proximal wall of the cap and the inside side walls of the cap. The porous element 630 can be expandable and compressible and can, in some embodiments, have an outer diameter that is larger than an inner diameter of the cap so that the porous element expands against the inside wall of the cap when coupled with or otherwise assembled with the cap. The porous element 630 can be coupled the cap so as to be positioned in contact with an inside surface of the proximal end of the cap.

In some embodiments, the porous element 630 can be positioned at a base of the elongate member 620, at a proximal end portion of the internal volume 610, and/or anywhere within the internal volume 610. For example and without limitation, in some embodiments, the porous element 630 can be positioned at a base of the elongate member 620 and can have an opening therein that can be sized and configured to pass over the elongate member 620 such that, when the porous element 630 is positioned in an operable position (e.g., at a proximal end of the internal volume 610), the elongate member 622 can extend through the opening in the porous element 630.

In some embodiments, the space occupied by or the volume of the porous element 630 can be a sub-portion of the internal volume 610. In other words, the porous element 630 can, in some embodiments, occupy less than the entire volume of the internal volume 610. In some embodiments, the porous element 630 can, in some embodiments, occupy 25% or approximately 25% of the volume of the internal volume 610 of the cap, or can, in some embodiments, occupy from 20% or approximately 20% or less than 20% to 50% or approximately 50% or more than 50% of the internal volume 610 of the internal volume 610 of the cap, or can, in some embodiments, occupy from 30% or approximately 30% to 40% or approximately 40% of the internal volume 610 of the internal volume 610 of the cap, or of any value within any of the foregoing ranges or from and to any values within any of the foregoing ranges, with the volume of the internal volume 610 of the cap being determined before a separate connector is advanced into the internal volume 610 (i.e., of the cap before it has been connected to a catheter). In some embodiments, the volume within the internal volume 610 is greater than a volume of liquid within the hub or connector that the elongate member 620 is configured to displace when the elongate member 620 is advanced into the hub or connector.

In any embodiments disclosed herein, the porous element 630 can be configured to absorb and retain liquid that that comes into contact with the porous element 630, at least until the porous element 630 becomes saturated with liquid. In some embodiments, the porous element 630 can have sponge-like characteristics. In this arrangement, the porous element 630 can absorb and retain liquid that is expelled from the hub of the catheter or transfer set as the cap is engaged with the hub to reduce the likelihood that liquid will be leaked from the cap as the cap is engaged with the hub of the catheter. Additionally, because the antimicrobial can be dissolved into the liquid as the cap is engaged with the hub, the liquid that can be absorbed by the porous element 630 can contain antimicrobial, so as to provide further disinfection capabilities to the porous element 630 and to other surfaces as any liquid, if any, is expelled from the porous element.

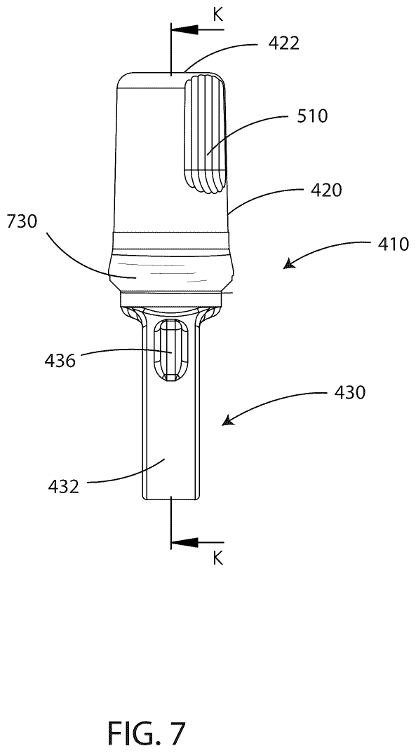

FIG. 7 is a side view of the embodiment of the cap combination set 410 containing a peritoneal dialysis cap 420 and base element 430, including one or more gripping portions 510 and a sealing band 730. As shown, any embodiments disclosed herein can include a scaling band 730 to secure the peritoneal dialysis cap 420 to the cap base element 430. The sealing band can retain the peritoneal dialysis cap 420 to the base element 430 prior to use or inhibit the detachment of the cap 420 from the base element 430 prior to use, and/or can prevent re-installation of the peritoneal dialysis cap 420 onto the base element 430 after the cap 420 has been detached from the base element 430 to prevent or inhibit the reuse of the cap 420 (which may not be sanitary). The peritoneal dialysis cap 420 can include a proximal end 422 and a distal end 424. The base element 430 can include a ring 432 sized to receive the finger of a user, the ring 432 having an opening 434 sized and configured to receive the finger of a user through the ring 432. In addition, in the embodiment depicted, the base element 430 can include a pair of side extensions 436, as described above. In some embodiments, the side extensions 436 can allow a user to exert a greater level of torque on the base element 430 when the base element 430 is positioned on a user's hand.

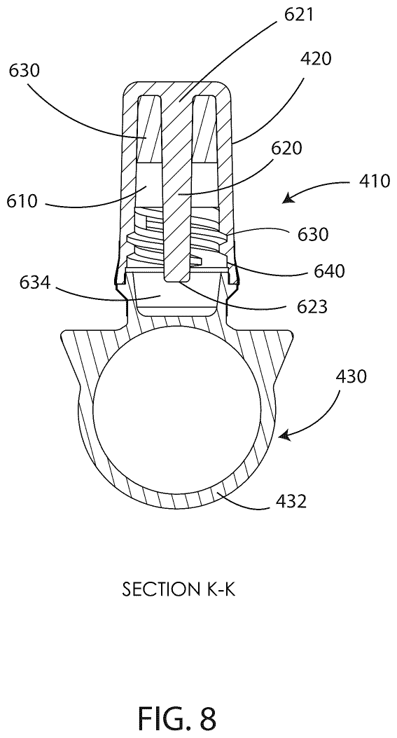

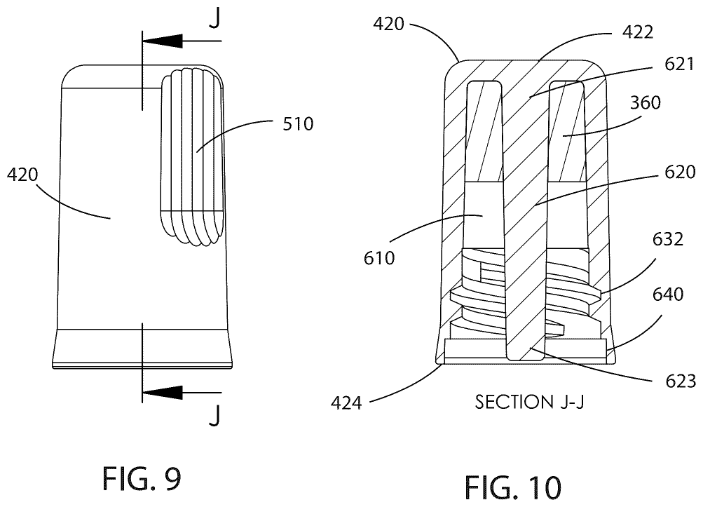

FIG. 8 is a cross-sectional view of the embodiment of the cap combination set 410 including the peritoneal dialysis cap 420 and base element 430 as shown in FIG. 5, taken along line K-K of FIG. 7. FIG. 9 is a side view of a peritoneal dialysis cap 420 in accordance with various embodiments herein, including showing a gripping portion 510. FIG. 10 is a side cross-sectional view of a peritoneal dialysis cap of FIG. 9, taken along line J-J as shown in FIG. 9. As illustrated in FIG. 10, in some embodiments, the diameter or horizontal cross-sectional width of the elongate member 620 can be larger than the horizontal cross-sectional width of the side wall(s) of the cap 420 and/or the vertical width of the proximal end 422 of the cap 420. For example, as shown, on at least one point along the elongate member, or along at least a majority of the vertical length of the elongate member 620, and/or along the entire vertical length of the elongate member 620, the diameter or horizontal cross-sectional width of the elongate member 620 can be larger than the horizontal cross-sectional width of some point and/or of every point along the vertical length of the side wall(s) of the cap 420, and/or of some point and/or of every point on the vertical width along the horizontal length of the proximal end 422 of the cap 420. As illustrated, the outer surface of the cap 420 can be tapered outwardly at the distalmost end of the cap 420.

As shown in many figures, in some embodiments, the elongate member 620 can be solid (not hollow) and/or can be formed unitarily with the side wall(s) of the cap 420 and/or the proximal end 422 of the cap 420, without providing separately formed components that are brought together. As shown, the elongate member 620 can be sufficiently long that it extends to about the distal end of the cap 420 and/or the elongate member 620 can be slightly shorter than the full vertical length of the side wall of the cap 420. By making the elongate member 620 shorter than the side wall of the cap 420, the elongate member 620 is less likely to be contaminated if a user (improperly) rests the cap 420 on a surface before use. Also, in some embodiments, when the elongate member 620 is shorter than the side wall of the cap 420, the cap 420 can be provided with a removable adhesively attached lid or seal (not shown) that extends across the distal opening of the side wall of the cap 420 to resist contamination of the interior of the cap 420 before use. As shown, the distalmost end of the threaded interior portion of the cap 420 can be proximal from the distalmost end of the side wall of the cap 420, and the portion of the side wall that is distal of the distalmost end of the threaded interior portion of the cap 420 can be thinner in horizontal cross-sectional width than horizontal cross-section of the portion of the side wall that is proximal from the threaded interior portion of the cap 420. This thinner portion can provide some degree of flexibility to enable slight movement or temporary radial expansion of the distal end of the cap 420 when attached to the hub 212 to assist in forming a seal between the cap 420 and the hub 212.

In some embodiments, as illustrated, the porous element 630 can surround the base of the elongate member 620 along at least a majority of the interior region between the base of the elongate member 620 and the proximal end of the threaded region on the interior of the cap 420. As shown, the vertical height or thickness of the porous element 630 can be at least as large as the distance between the proximal and distal ends of the threaded region of the cap 420. In some embodiments, as shown, the fluid absorption volume of the porous element 630 can be at least as great as the volume of the elongate member 420 to enable the porous element 630 to absorb any fluid that is displaced by insertion of the elongate member 420 into a hub 212 that is already filled with fluid.

Some embodiments of the peritoneal dialysis cap 420 can include an internal volume 610 through which an elongate member 620 can extend. The internal volume 610 can extend from a proximal end 422 of the body 421 of the cap 420 to a distal end 424 of the body 421 of the cap 420. As mentioned, the elongate member 620 can extend through a longitudinally axial centerline of the body 421 of the cap 420 and the internal volume 610 of the cap 420.

The elongate member 620 can have a proximal end 621 adjacent to the proximal end 422 of the cap 420 and can extend to a distal end 623 along a longitudinally axial centerline of the internal volume 610. In some embodiments, the elongate member 620 can have a round cross-sectional shape, as shown, or any other desired cross-sectional shape, such as a star shape, hexagonal shape, octagonal, or other polygonal shape. The elongate member 620 can be uniform along a length thereof, or can taper along all or a portion of a length thereof such that a diameter or cross-sectional size of the elongate member 620 at the distal end 623 thereof is less than a diameter or cross-sectional size of the elongate member 620 at the proximal end 621 thereof. In some embodiments, the elongate member 620 can extend to a length or to a point or plane that is less than a length of the cap 420 at a distal end 424 thereof so that the distal end 623 of the elongate member 620 does not extend past the distal end 424 of the cap 420.

In some embodiments, the elongate member 620 can be integrally (i.e., monolithically) formed with the body 421 of the cap 420. In some embodiments, the elongate member 620 can be separately formed and coupled with the body 421 of the cap 420.

In some embodiments, a porous element 630 can be positioned at the proximal end of the internal volume 610 of the peritoneal dialysis cap 420. In addition, at the distal end of the peritoneal dialysis cap 420 is a is an overlapping region 640 that has a flange that engages the base element 430. The base element 430 in the depicted configuration can include a recess 634 into which the proximal end 622 of the elongate member can extend.

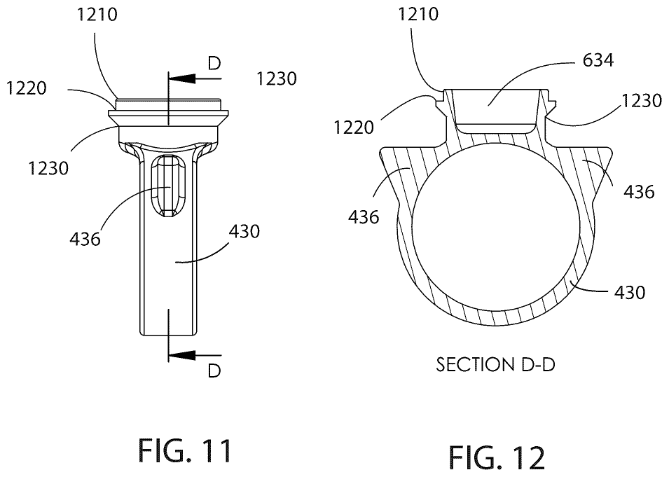

FIG. 11 is a side view of an embodiment of a peritoneal dialysis base element 430. FIG. 12 is a side cross-sectional view of the peritoneal dialysis base element 430 of FIG. 11, taken along line D-D shown in FIG. 11. The base element 430 can include a ring 432 sized to receive the finger of a user, with a hole 434 through the ring 432. The base element 430 further can include a recess 634 into which the distal end 623 of the elongate member 620 (not shown) can extend into when the cap 420 is coupled with the base element 430. In some embodiments, the base element 430 can include an upper flange surface 1210 and a lower flange surface 1220, along with an outer flange surface 1230.

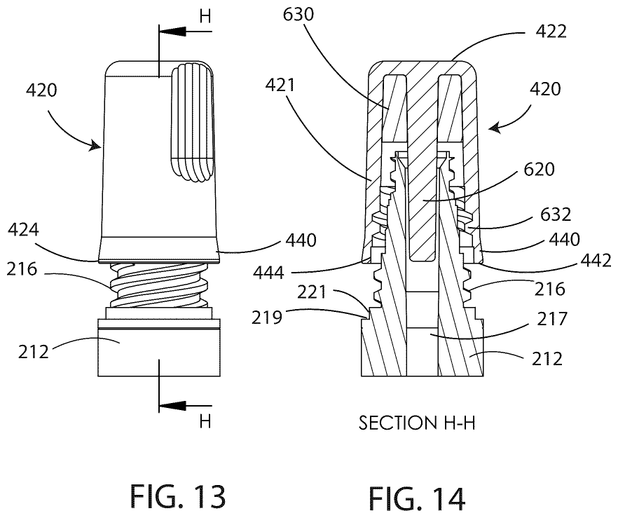

FIG. 13 is a side view of an embodiment of a peritoneal dialysis cap 420 being installed on a proximal hub 212 of a transfer set. FIG. 14 is a cross-sectional view of the embodiment of the peritoneal dialysis cap 420 shown in FIG. 13 being installed on the proximal hub 212 of the transfer set, taken along line H-H shown in FIG. 13. As the cap 420 is being advanced into engagement with the hub 212, the cap 420 can be rotated so that the threads 632 of the cap 420 rotate to engage the threads 216 of the proximal hub 212 of a transfer set. As the cap 420 is being advanced into engagement with the hub 212, the elongate member 620 can extend into a passageway 217 of the proximal hub 212 of the transfer set. The elongate member can project into the interior or passageway 217 of the proximal hub 212 of the transfer set as the body 421 of the cap 420 moves into position over threads 216 on the proximal hub 212 of the transfer set.

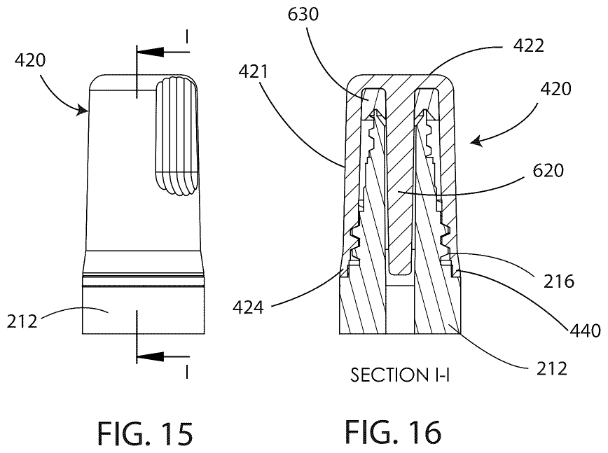

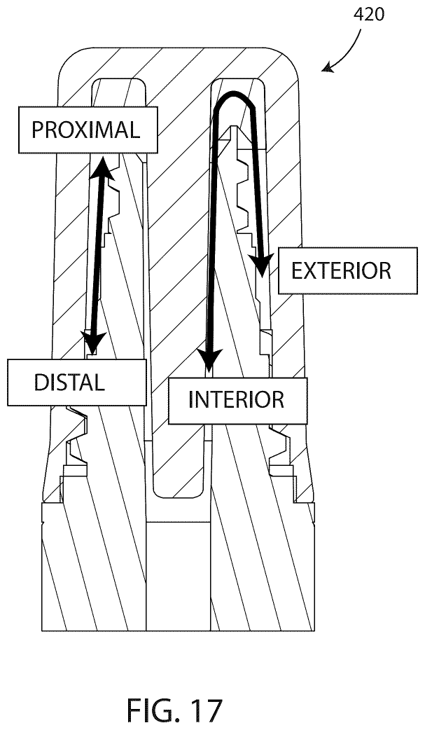

FIG. 15 is a side view of an embodiment of a peritoneal dialysis cap 420 installed on an exemplifying proximal hub 212 of a transfer set. FIG. 16 is a cross-sectional view of the embodiment of the peritoneal dialysis cap 420 installed on the proximal hub 212 of the transfer set, taken along line I-I as shown in FIG. 15. FIG. 17 cross-sectional view of an embodiment of the peritoneal dialysis cap 420 installed on a transfer set in accordance with various embodiments herein, showing proximal/distal orientations and interior/exterior orientations. The elongate member 620 on the peritoneal dialysis cap 420 can extend into the lumen of the transfer set, and the threads 632 of peritoneal dialysis cap 420 can engage the corresponding threads 216 of the transfer set.

In any embodiments disclosed herein, the cap can have one or more sealing surfaces, or two or two or more sealing surfaces configured to provide a liquid-tight seal between the cap and the medical connector that the cap is engaged with when the cap is engaged with the medical connector. In some embodiments, the one or more sealing surfaces can be configured to seal against an outer surface (such as an outer diameter) of the hub of the catheter or transfer set. In some embodiments, the one, two, or more sealing surfaces can be located distal to the threads and can provide a liquid-tight seal between the cap and the medical connector when the cap is installed on the medical connector.

In some embodiments, the cap 420 can have an overlapping region 640 at a distal end of the cap 420 that can overlap one or more portions or surfaces of the hub of the catheter or transfer set. In some embodiments, the overlapping region 640 can have a sealing flange 440 that engages the base element 430 and/or one or more surfaces of the hub of a catheter or the transfer set. Some embodiments of the cap 420 can have one or more sealing surfaces at a distal end 424 of the body 421 of the cap 420.

For example and without limitation, with reference to FIGS. 13-16, some embodiments of the cap 420 can have a sealing flange 440 at a distal end 424 of the body 421 of the cap 420. In some embodiments, the sealing flange 440 can have a first or distal sealing surface 442 that can be configured to engage with and seal against a first surface 219 of the proximal hub 212 of a transfer set. In some embodiments, the first or distal sealing surface 442 can be configured to abut against the first surface 219 in an axial direction as the cap 420 is threadedly engaged with the proximal hub 212 to create a seal against the first surface 219 of the proximal hub 212 of the transfer set. In some embodiments, the first sealing surface 442 can have a planar end surface that is approximately perpendicular to a longitudinally axial centerline of the cap 420 and that is configured to abut against the first surface 219 of the proximal hub 212 of the transfer set.

In some embodiments, the sealing flange 440 can have a second or radial scaling surface 444 that can be configured to engage with and seal against a second surface 221 of the proximal hub 212 of a transfer set. In some embodiments, the second or radial sealing surface 444 can be configured to constrict or squeeze in a radially inward direction against the second surface 221 to create a seal against the second surface 221 of the proximal hub 212 of the transfer set. For example and without limitation, in some embodiments, the sealing flange 440 can be configured to create an interference fit relative to the second surface 221 of the proximal hub 212 by have an inner diameter that is less than an outer diameter of the second surface 221 of the proximal hub 212. In some embodiments, without limitation, the sealing flange 440 can be configured to create an interference fit relative to the second surface 221 of the proximal hub 212 by have an inner diameter that is 5% or approximately 5% less than, or 10%, approximately 10%, or more than 10% less than, or from 2% or approximately 2% to 15% or approximately 15% less than an outer diameter of the second surface 221 of the proximal hub 212.

As illustrated in FIG. 16, in some embodiments the cap 420 and/or at least the threaded region of the interior of the cap 420 can be substantially rigid, and not generally elastomeric, resilient, and/or flexible, such that the threaded region does not change shape and/or expand during or after attachment to the hub 212. When the cap 420 is attached to the hub 212, the cap 420 can form a seal against the hub 212 by tightly, snuggly, forcible, securely, and/or frictionally contacting the hub 212 in a radial direction and/or in a longitudinal direction as shown. For example, in some embodiments, the cap 420 can be configured to resist venting at a normal fluid or air pressure within the hub 212 that is within the range of expected pressure of a peritoneal dialysis or hemodialysis catheter in normal use, such as an average blood pressure of a patient. In some embodiments, the pressure-resistance of the cap 420 when attached to the hub 212 can be substantially higher, such as at least about 10 psi, or at least about 20 psi, to provide increased protection against leaking when attached.

Any embodiments of the cap disclosed herein can be configured such that no seal or obstruction is created against an inside surface of the hub or connector (such as an inner passageway) that the cap is engaged with, even when the cap is fully engaged with the hub or connector. Any embodiments of the cap can be configured such that the cap only seals against an outside or outwardly facing surface of the hub or connector that the cap engages with, even when the cap is fully engaged with the hub or connector. In some embodiments, the cap can be configured such that there are no sealing surfaces at or adjacent to a proximal end portion of the elongate member. In some embodiments, the cap can be configured such that all sealing surfaces or seals are positioned at or adjacent to a distal portion of the cap, adjacent to an opening in the distal end of the cap.

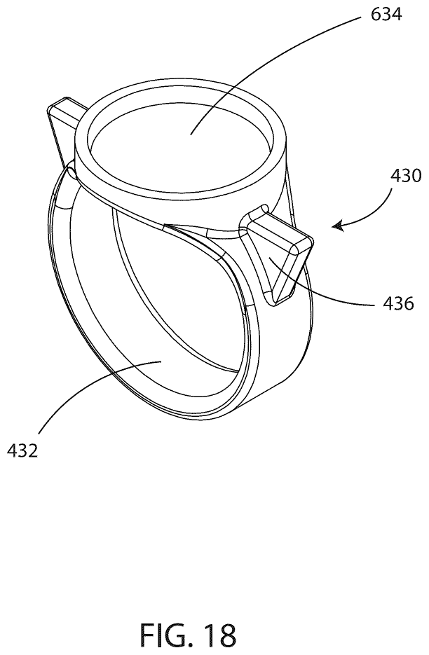

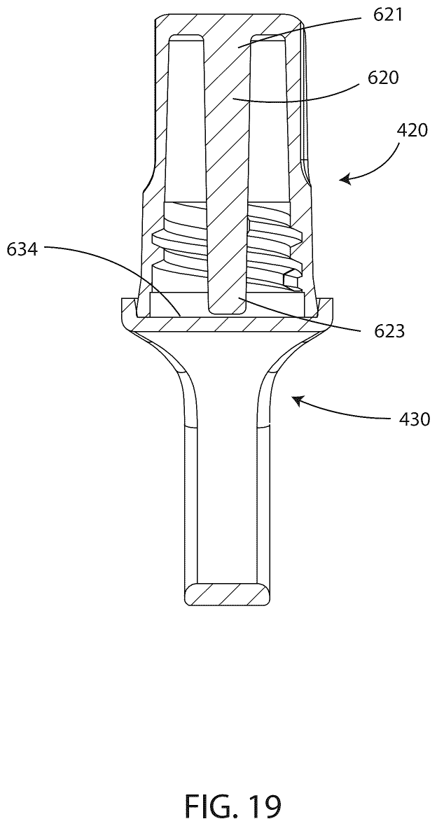

FIG. 18 is a perspective view of an embodiment of a peritoneal dialysis base element 430, having a ring 432, side extensions 436, and a recess 634. FIG. 19 is a cross-sectional view of an embodiment of a peritoneal dialysis cap 420 and the embodiment of the base element 430 shown in FIG. 18 through a longitudinally axial centerline of the base element 430. In any embodiments of the base element disclosed herein (including, without limitation, base element 430 shown in any of the FIGS. 11-12 and 18-20), the base element can have any of the features, components, materials, or other details of any other embodiments of base elements disclosed herein, in place of or in combination with any of the features, components, materials, or other details of the embodiment of the base element (including, without limitation, base element 430). In some embodiments, the distal end of the cap 420 and/or the elongate member 620 can extend into the recess 634 of the base element 430.

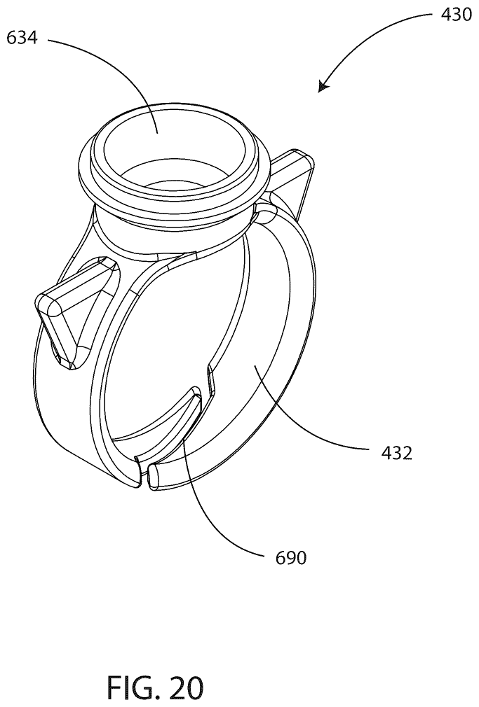

FIG. 20 is a perspective view of another embodiment of a peritoneal dialysis base element 430, showing the ring 432 with a split region 690 in the ring 432. In some embodiments, the split region 690 in the ring 432 can allow the ring to expand (e.g., increase) and contract (e.g., decrease) in size to accommodate different sized fingers of users.

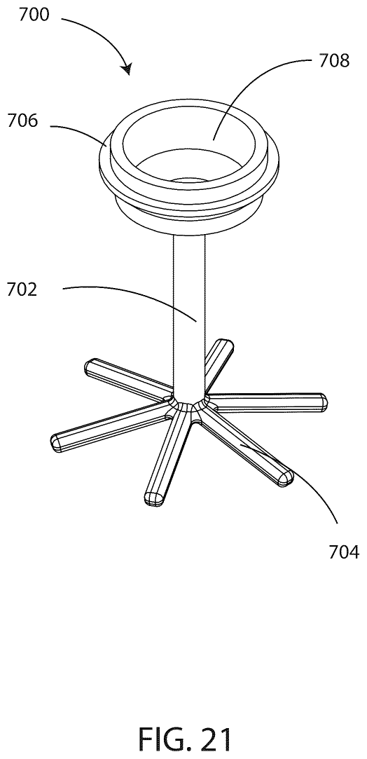

FIG. 21 is a perspective view of another embodiment of a peritoneal dialysis base element 700. In any embodiments of the base element disclosed herein (including, without limitation, the embodiment of the base element 700 shown in FIG. 21), the base element can have any of the features, components, materials, or other details of any other base element embodiments disclosed herein, in place of or in combination with any of the features, components, materials, or other details of the embodiment of the base element (including, without limitation, base element 700). With reference to FIG. 21, some embodiments of the base element 700 can have a shaft 702 and a multi-prong retaining region 704 that can be grasped by the user. The base element 700 can have an annular flange or ledge 706 that is sized and configured to receive the distal end portion of the cap, and a recess 708 formed in an end of the base element 700 into which the distal end of an elongate member of the cap can extend.

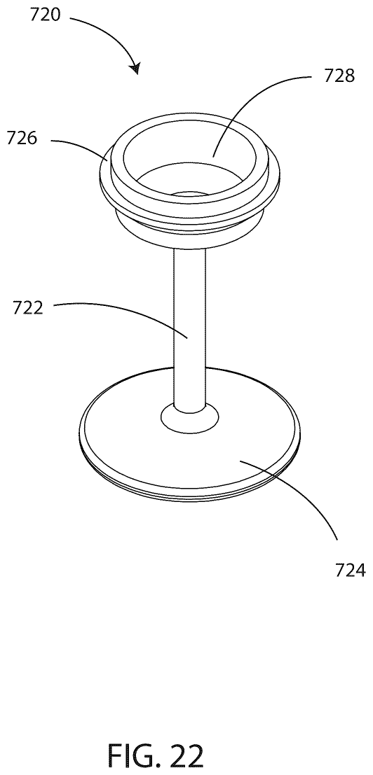

FIG. 22 is a perspective view of another embodiment of a peritoneal dialysis base element 720. In any embodiments of the base element disclosed herein (including, without limitation, the embodiment of the base element 720 shown in FIG. 22), the base element can have any of the features, components, materials, or other details of any other base element embodiments disclosed herein, in place of or in combination with any of the features, components, materials, or other details of the embodiment of the base element (including, without limitation, base element 720). With reference to FIG. 22, some embodiments of the base element 720 can have a shaft 722 and a circular or disk shaped retaining region 724 that can be grasped by the user. The base element 720 can have an annular flange or ledge 726 that is sized and configured to receive the distal end portion of the cap, and a recess 728 formed in an end of the base element 720 into which the distal end of an elongate member of the cap can extend.

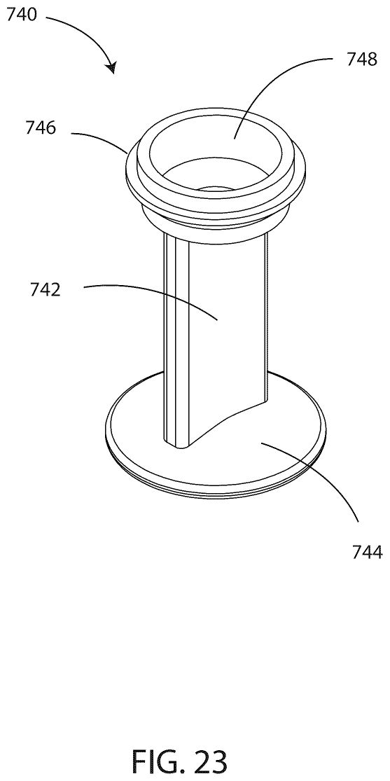

FIG. 23 is a perspective view of another embodiment of a peritoneal dialysis base element 740. In any embodiments of the base element disclosed herein (including, without limitation, the embodiment of the base element 740 shown in FIG. 23), the base element can have any of the features, components, materials, or other details of any other base element embodiments disclosed herein, in place of or in combination with any of the features, components, materials, or other details of the embodiment of the base element (including, without limitation, base element 740). With reference to FIG. 23, some embodiments of the base element 740 can have a shaft 742 and a circular or disk shaped retaining region 744 that can be grasped by the user. The shaft 742 can have a generally rectangular shaped cross-section. The base element 740 can have an annular flange or ledge 746 that is sized and configured to receive the distal end portion of the cap, and a recess 748 formed in an end of the base element 740 into which the distal end of an elongate member of the cap can extend.

In any embodiments disclosed herein, the cap can include an antimicrobial agent, for example and without limitation, chlorhexidine acetate. Other antimicrobial agents that can be used with any embodiments disclosed herein can include chlorhexidine base, chlorhexidine acetate, chlorhexidine gluconate, EDTA, silver sulfadiazine, or Taurolidine, or combinations thereof. Other suitable antimicrobial agents can also be used with any cap embodiments disclosed herein. The term “antimicrobial,” as used here, can include any substance or substances that kills or inhibits the growth of organisms such as bacteria, fungi, protozoa, viruses, etc. It should also be noted that there can be one or more antimicrobial agents used in some embodiments disclosed herein. Therefore, throughout this document, the term antimicrobial or antimicrobial agent should be understood to refer to one or more antimicrobial agents.

In any embodiments disclosed herein, the antimicrobial agent can be coated on the elongate member and/or on other surfaces of the cap, such as an inside surface of the body of the cap, and/or a proximal wall of the cap. As such, the antimicrobial agent can be delivered as a coating that elutes from a coated elongate member, that is coated on, or impregnated into, an elongate member (such as 250 μg or approximately 250 μg of chlorhexidine acetate in a layer 2 μm or approximately 2 μm thick along a length of the elongate member, or as 50 μg or approximately 50 μg of chlorhexidine acetate in a layer that is 0.4 μm thick or approximately 0.4 μm thick). Antimicrobial agent from the cap can dissolve into the displaced fluid, thereby disinfecting the proximal end of the catheter. In this arrangement, the antimicrobial can be transferred from the cap to the solution within the catheter. The antimicrobial substance or agent from the cap can dissolve into the catheter fluid, thereby disinfecting at least the proximal end of the catheter. Furthermore, in some embodiments, when the catheter fluid dries, the catheter fluid can leave deposits or coatings of chlorhexidine acetate or other appropriate antimicrobial on the cap and/or catheter hub.

In some embodiments, the elongate member can be configured to displace a volume of fluid from within the catheter as the elongate member is advanced into the hub of the catheter. In some embodiments, the volume of fluid that can be displaced can equal or approximately the volume of the coated or uncoated elongate member. In some embodiments, the volume of fluid that can be displaced can equal or approximately equal the volume of the coated or uncoated elongate member that extends past the porous element in the distal direction, or the volume of the coated or uncoated elongate member minus the solid or displacement volume of the porous element.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A capping system for a medical connector, comprising:
a base element comprising:
a finger retainer on a distal end of the base element, the finger retainer including a hole extending through the finger retainer; and
a recess positioned on a proximal end of the base element and above the finger retainer; and
a cap comprising:
a body having a closed proximal end and a distal end;
an interior volume within the body;
an elongate member extending from the closed proximal end of the body axially through at least a portion of the interior volume surrounding the elongate member to a distal end of the elongate member, the elongate member comprising an antimicrobial; and
threads for securing the cap to the medical connector;
wherein at least a portion of the proximal end of the base element is configured to be secured to the distal end of the cap.

2. The capping system of claim 1, wherein the recess is configured to receive at least a portion of the distal end of the elongate member.

3. The capping system of claim 1, wherein the cap comprises a heat shrink band at the distal end of the cap providing a barrier to the interior volume.

4. The capping system of claim 3, wherein the cap is secured to the base element by the heat shrink band to prevent detachment of the cap from the base element.

5. A method for retaining a cap on a base element for a medical connector, comprising:
obtaining the base element containing a finger retainer on a distal end of the base element, a hole extending through the finger retainer, and a recess at a proximal end of the base element;
attaching the base element to a distal end of the cap, the cap including a partially enclosed interior space and a proximal end; and
securing the cap to the base element at a distal end of the cap.

6. The method of claim 5, further comprising removing the cap from the base element such that a heat shrink band is retained on the base element.

7. The method of claim 6, wherein the heat shrink band is configured to prevent reinstallation onto the base element.

8. The method of claim 5 further comprising removing the cap from the base element to prevent installation onto the base element to prevent reuse of the cap.

9. The method of claim 5, further comprising placing the base element on a finger of a user through the hole.

10. The method of claim 9, wherein the base element comprises one or more side extensions configured to allow the user to exert a greater torque force on the base element by restricting a rotation of the base element placed on the finger.

11. A base element for use with a cap for a medical connector, the base element comprising:
a finger support portion having a hole therethrough that is sized and configured to receive a finger of a user; and
a cap support portion coupled with the finger support portion and extending away from an outside surface of the finger support portion, the cap support portion being configured to removably couple with the cap for the medical connector;
wherein:
the cap support portion comprises an annular wall and a space that is surrounded by the annular wall; and
the space is configured to receive at least a portion of the cap in the space when the cap is secured to the base element.

12. The base element of claim 11, further comprising one or more side extensions extending away from the outside surface of the finger support portion, the one or more side extensions configured to enable the user to exert a greater torque force on the base element by pressing on the one or more side extensions with fingers of a user.

13. The base element of claim 12, wherein the one or more side extensions have a rounded or triangular shape.

14. The base element of claim 11, wherein the annular wall of the cap support portion comprises an outwardly facing surface configured to engage with an inner surface of a sealing flange of the cap positioned at a distal end the cap.

15. The base element of claim 11, wherein the space surrounded by the annular wall includes a radially inward facing surface and a bottom surface, the space being configured to receive at least a distal end of an elongate member of the cap within the space when the cap is coupled with the base element.

16. The base element of claim 11, wherein the cap support portion of the base element comprises an annular flange extending outwardly from an outside surface of the annular wall of the cap support portion, wherein an upper flange surface is configured to engage a distal end of a sealing flange of the cap.

17. The base element of claim 11, further comprising a sealing band positioned on an outer surface of the base element at a proximal end thereof, the sealing band configured to retain the cap and inhibit detachment of the cap from the base element prior to use.

18. The base element of claim 17, wherein the sealing band is configured to prevent re-installation of the cap onto the base element to prevent reuse of the cap.

19. The base element of claim 11, wherein an axial centerline of the hole of the finger support portion is perpendicular to an axial centerline of the space surrounded by the annular wall.

20. The base element of claim 11, wherein the cap is a peritoneal dialysis cap.
